(19) United States
(12) Patent Application Publication
(10) Pub. No.: US 2011/0311963 A1
(43) Pub. Date: Dec. 22, 2011

(54) Method and Apparatus for Addressable Flow Cells in Single Molecule Sequencing

(75) Inventors: W. MICHAEL LAFFERTY, ENCINITAS, CA (US); JOSEPH BEECHEM, EUGENE, OR (US); HONGYE SUN, BELMONT, CA (US); MICHAEL METZKER, HOUSTON, TX (US)

(73) Assignee: LIFE TECHNOLOGIES CORPORATION, CARLSBAD, CA (US)

(21) Appl. No.: 13/049,660

(22) Filed: Mar. 16, 2011

Related U.S. Application Data
(60) Provisional application No. 61/314,542, filed on Mar. 16, 2010.

(51) Int. Cl. C12Q 1/68

(52) U.S. Cl. 435/6.1

(57) ABSTRACT

A method of sequencing a plurality of template nucleotide sequences includes immobilizing the plurality of template nucleotide sequences on a substrate. A first subset of the plurality of template nucleotide sequences is immobilized in a first field of view and a second subset of the plurality of template nucleotide sequences is immobilized in a second field of view. The first and second subsets are hybridized to a caged primer. The caged primer includes a caging group. The method further includes lysing the caging group from the caged primer in the first field of view and observing the first field of view to detect sequencing of the first subset of the plurality of template nucleotide sequences.

### CROSS-REFERENCE TO RELATED APPLICATION(S)

This application claims the benefit of U.S. Provisional Application No. 61/314,542 filed Mar. 16, 2010, which is herein incorporated by reference in its entirety.

### FIELD OF THE DISCLOSURE

The disclosure generally relates to addressable flow cells used in single molecule sequencing (“SMS”). More specifically, the disclosure relates to using flow cells with addressable fields of view which can be used for multiple reads on one or more DNA sequences.

### BACKGROUND

Conventional detection of asynchronous single molecule sequencing (“SMS”) utilizes detectors (e.g., cameras) to record signals from all reaction sites simultaneously. The reaction sites are dispersed over the surface of the substrate under study. Thus, signal collection is limited to a single field of view (“FOV”) that encompasses the entire substrate over which the reaction sites are dispersed. Such conventional detection limits the size of useful substrates and the number of reactions that can be observed. In addition, such conventional detection provides low resolution of observed reactions, leading to complex computational routines to reconcile observations and greater error.

As such, an improved SMS detection system would be desirable.

### SUMMARY

In a first aspect, a method of sequencing a plurality of template nucleotide sequences includes immobilizing the plurality of template nucleotide sequences on a substrate. A first subset of the plurality of template nucleotide sequences is immobilized in a first field of view and a second subset of the plurality of template nucleotide sequences is immobilized in a second field of view. The first and second subsets are hybridized to a caged primer. The caged primer including a caging group. The method further includes lysing the caging group from the caged primer in the first field of view and observing the first field of view to detect sequencing of the first subset of the plurality of template nucleotide sequences.

In a second aspect, a method of sequencing a template nucleotide sequences includes applying a caged primer to the template nucleotide sequences. The caged primer includes an oligonucleotide primer and a caging group coupled to the oligonucleotide primer. The method further includes lysing the caged group from the caged primer.

In a third aspect, a kit to sequence template nucleotide sequences includes a substrate to immobilize template nucleotide sequences. The substrate is sized to include a plurality of fields of view. The kit also includes a caged primer including an oligonucleotide primer and a caging group coupled to a terminal end of the oligonucleotide primer.

In a fourth aspect, a device includes a substrate to immobilize a plurality of target nucleotides. A target nucleotide of the plurality of target nucleotides is hybridized to a caged primer. The caged primer includes a caging group. The substrate is sized to provide a plurality of fields of view. The device further includes a radiation source to selectively address fields of view of the plurality of fields of view. The radiation source has a peak wavelength to lyse the caging group from the caged primer in a select field of view of the plurality of fields of view. The device also includes a detector to detect incorporation of a nucleotide to the target nucleotide in the select field of view.

In a fifth aspect, a method of sequencing a template nucleotide sequence includes applying a primer to the template nucleotide sequence. The primer includes an oligonucleotide hybridized to a portion of the template nucleotide sequence. The method further includes incorporating a caging nucleotide including a caging group to a terminal end of the oligonucleotide hybridized to the portion of the template nucleotide.

### BRIEF DESCRIPTION OF THE DRAWINGS

The present disclosure may be better understood, and its numerous features and advantages made apparent to those skilled in the art by referencing the accompanying drawings.

FIG. 1 includes an illustration of a method of sequencing a template nucleotide sequence.

FIG. 2 and FIG. 3 include illustrations of exemplary caging groups coupled to a nucleotide.

FIG. 4 and FIG. 5 include illustrations of exemplary immobilized templates.

FIG. 6 includes an illustration of an exemplary sequencing device.

FIG. 7 includes an illustration of an exemplary kit.

FIG. 8 and FIG. 9 include illustrations of exemplary template sequences.

FIG. 10 includes a graph illustration of a change in mass resulting from release of the caging group.

FIG. 11, FIG. 12, FIG. 13, FIG. 14, FIG. 15, FIG. 16, FIG. 17, FIG. 18, FIG. 19, FIG. 20, FIG. 21, and FIG. 22 include illustrations of fluorescent responses to nucleotide incorporation.

The use of the same reference symbols in different drawings indicates similar or identical items.

### DESCRIPTION OF THE PREFERRED EMBODIMENT(S)

Embodiments include methods of detecting nucleotide incorporation and devices for performing such methods. The methods include activating sequencing reactions in a select field of view on a substrate that defines multiple fields of view and observing the selected field of view to detect nucleotide incorporation. The multiple fields of view can be located within a flow cell. Sequencing reactions can be activated in a second select field of view and the second field of view can be observed to detect nucleotide incorporation. In an example, activating sequencing reactions within a field of view can include cleaving or lysing a caging group from a caged primer coupled to a template nucleotide sequence, permitting enzymatic incorporation of nucleotides complementary to the template nucleotide sequence to proceed.

With such a method, exemplary devices can include substrates sized to include multiple reactions sites, each associated with a field of view. The multiple reaction sites can be within a single flow cell. A reaction site can be activated and the associated field of view observed. Subsequently, another reaction site can be activated and its associated field of view observed. In a particular example, a reaction site can be activated to initiate sequencing of a template nucleotide sequence, such as a gene or genetic fragment, and the sequencing reactions can be observed to determine the nucleotide sequence of the template.

An advantage of the present technique is the flexibility to choose how long a given field of view (“FOV”) is viewed. In an example, the length of viewing can correspond to the read length. In an example, a device provides multiple reaction sites, each reaction site defining a different FOV. The reaction sites can be activated independent of each other and the length of viewing for each reaction site can be determined by the operator or as a function of different factors. In a particular example, a suitable surface for single molecule sequencing (“SMS”) is provided and a DNA template is attached to the surface. The template can be primed with a primer having a terminator, such as a caging group, thereon. In an example, the terminator can prevent incorporation of additional nucleotides on the template. For example, the terminator can limit enzyme activity, preventing incorporation of a subsequent nucleotide. The terminator can be a photocleavable terminator removable by appropriate radiation.

To activate a site after it is captured in the FOV, radiation energy (e.g., ultraviolet radiation) can be provided to the site to remove the terminator (e.g., caging group) from the primer. Once removed, sequencing begins and is detected in the FOV under study. The operator can maintain the FOV for a desired duration or while the sequencing process is pending. Thereafter, another site can be activated and the process repeated. With such a process, the same template, such as the same DNA template, can be sequenced several times under multiple FOVs. Such a technique can also be applied to multiple DNA samples on the same substrate. In another example, similar templates can be sequenced under different conditions by changing conditions when observing different fields of view.

In an example where a single DNA is sequenced, a single large flow cell can be used. The surface area of the flow cell can define many FOVs (e.g., hundreds or thousands FOVs). The surface of the flow cell can have DNA templates attached thereto and ready to be sequenced. An area, which can approximate the size of a single FOV, can be activated independently from the remaining surface area. Such an area defines a region used for the first set of SMS readings.

In an example, the activation can be implemented by delivering a quantity of sequencing reaction mix only to the region of the surface under study. The quantity of sequencing reaction mix can be delivered by control flow (e.g., digital fluidics such as Raindance.) In another example, activation is implemented optically by photocleaving a caging group from a caged nucleotide or a caged primer terminus. In a further example, optical activation is implemented through an optically reversible terminator used as a trigger.

Once a region is activated, the sequencing reaction occurs in real time within the region while the remainder of the surface area of the flow cell remains inactive. Once the desired amount of sequencing data is harvested, a subsequent region of the flow cell surface can be activated, and the sequencing cycle can be repeated. An advantage of the present process is the flexibility to select shorter or longer read lengths. Different sites can be viewed for shorter or longer duration independent of each other. Thus, a larger number of individual sites can be read with short reaction time at each site, a smaller number of individual sites can be read with longer reaction or read time at each site, or a combination thereof can be performed.

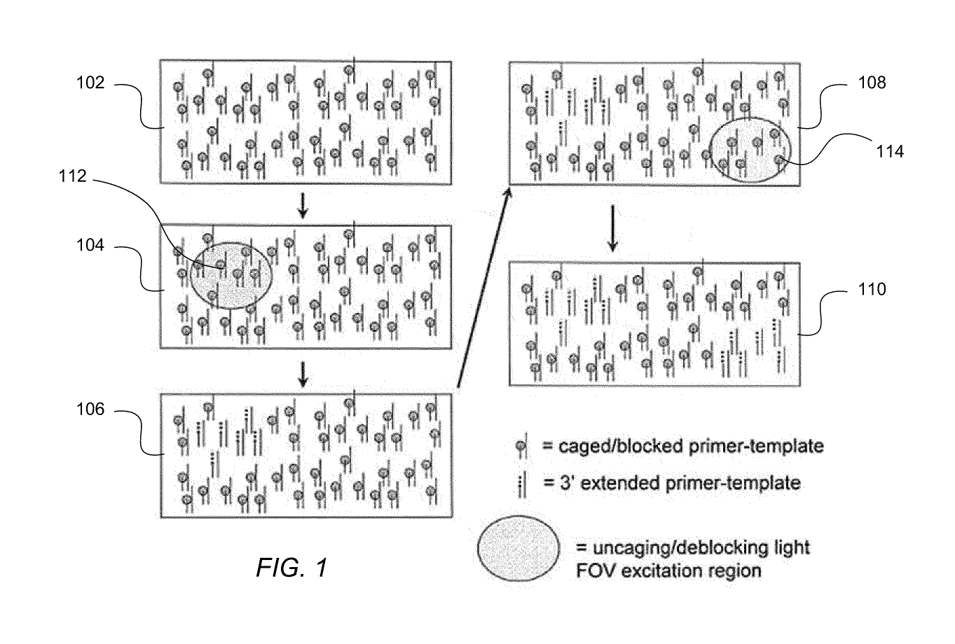

FIG. 1 schematically illustrates an implementation according to an example. Specifically, FIG. 1 is a schematic representation of multiple FOVs asynchronous sequencing using photocleavable caged/blocked primer. Reagents to support sequencing by synthesis are present over the sequencing surface. However, primer/templates exposed to photoactivation light support synthesis that is monitored in real-time.

As illustrated at 102 of FIG. 1, templates are immobilized on a substrate and are hybridized with a caged primer. The caged primer includes a nucleotide series, such as an oligonucleotide, and a caging group, also referred to as a blocking group. In particular, the caging group can be coupled to a terminal nucleotide of the oligonucleotide, such a 3′ end terminal nucleotide or a 5′ end terminal nucleotide. For example, the caging group can be coupled to a 3′ end terminal nucleotide.

In an example, the caging group is a photocleavable or photolysable caging group. Such caging groups can be cleaved or lysed when exposed to electromagnetic radiation having wavelengths between 100 nm and 1000 nm. In particular, the electromagnetic radiation can have a peak wavelength in the visible spectrum or in the ultraviolet spectrum. For example, the electromagnetic radiation can have a peak wavelength of not greater than 500 nm, such as not greater than 400 nm, or even not greater than 360 nm. In particular, the electromagnetic radiation can have a peak wavelength in a range of 100 nm to 1000 nm, such as a range of 100 nm to 500 nm, a range of 100 nm to 400 nm, a range of 200 nm to 360 nm, such as a range of 210 nm to 325 nm.

An exemplary caging group can have nitrobenzyl functionality. For example, an exemplary caging group can include nitrobenzyl, α-carboxy-2-nitrobenzyl (CNB), 1-(2-nitrophenyl)ethyl (NPE), 4,5-dimethoxy-2-nitrobenzyl (DMNB), 1-(4,5-dimethoxy-2-nitrophenyl)ethyl (DMNPE), or 5-carboxymethoxy-2-nitrobenzyl (CMNB) caging groups, or any combination thereof. In an example, the caging group includes a nitrobenzyl caging group. In an additional example, the caging group includes an α-carboxy-2-nitrobenzyl (CNB) caging group. In another example, the caging group includes 1-(2-nitrophenyl)ethyl (NPE) caging group. In a further example, the caging group includes a dimethoxy nitrobenzyl caging group, such as 4,5-dimethoxy-2-nitrobenzyl (DMNB), 1-(4,5-dimethoxy-2-nitrophenyl)ethyl (DMNPE) caging groups, or any combination thereof.

In particular, a variety of caged compounds with different characteristics are available, as identified at Table 1.

TABLE 1
Properties of Different Caged Groups

| | | | | Confers | Long- |
|---|---|---|---|---|---|
| | | Photolysis | Inertness of | Water | Wavelength |
| Caging | Uncaging | Quantum | Photolysis | Solu- | Absorption |
| Group | Rate | Yield | By-product | bility | (≧360 nm) |
| CNB | ++++ | +++++ | +++++ | +++++ | ++ |
| NPE | +++ | +++ | +++ | + | ++ |
| DMNPE | +++ | +++ | +++ | + | +++++ |
| DMNB | +++ | +++ | ++ | + | +++++ |
| CMNB | +++ | +++ | + | ++++ | +++ |
| NP | +++ | +++ | + | + | ++ |

In Table 1, “+++++” denotes optimal response and “+” denotes a poor response. The structures of nitrobenzyl moiety and the atom to which it is attached have some effect on the efficiency and wavelength for uncaging (e.g., cleaving or lysing).

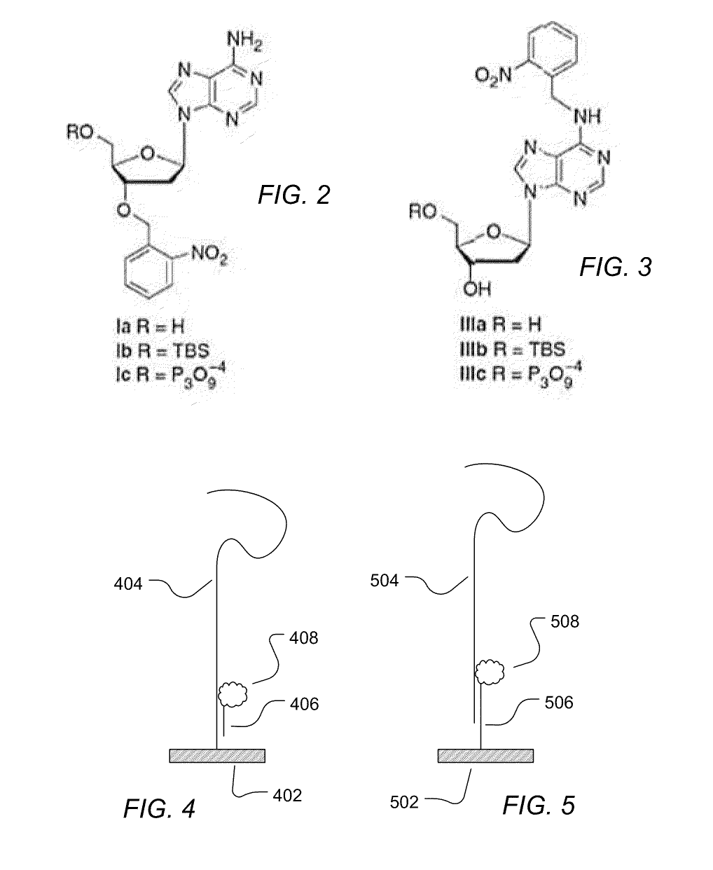

The caging group can be coupled to a nucleotide. For example, the caging group can be coupled to a nucleotide on the base, the sugar, or a phosphate or sulfate group. As illustrated in FIG. 2, a nitrobenzyl caging group is coupled to the 3-OH group of the nucleotide sugar. Such a caging group can be referred to as a true light terminator. True light terminators can be added to an oligonucleotide primer by using standard oligonucleotide synthetic chemistry. In another example illustrated in FIG. 3, a nitrobenzyl group is coupled to a base of a nucleotide, referred to as a virtual terminator. Such a compound helps labeling the primer in situ which prevents degradation.

The caging group coupled nucleotide (“caging nucleotide”) can be incorporated into an oligonucleotide primer, forming a caged primer. Alternatively, the caging nucleotide can be incorporated at a terminal end of a primer hybridized to a template nucleotide sequence, preventing further nucleotide incorporation. For example, the primer can be extended to include the caging nucleotide using polymerase in situ. The caging nucleotide can include the caging group either on the base or on the 3-OH group. If the caging nucleotide is incorporated at a 3′ end of the primer, such a caging nucleotide prevents further extension of the primer.

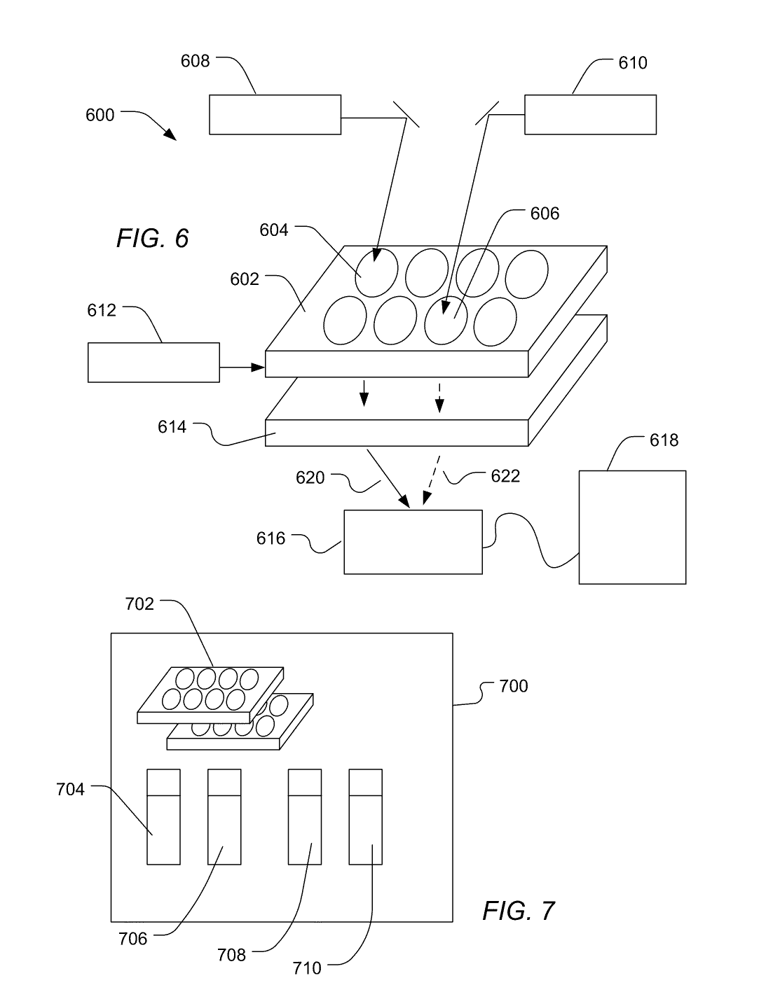

Returning to FIG. 1, the template/caged primer hybrids are immobilized to a surface of a substrate. In an example, the template can be immobilized directly to the substrate using terminal functionality attached to the template. For example, the template can be biotinylated and, for example, coupled to an avidin group, such as strepavidin, or can be coupled to a polymeric group, such as polyethylene glycol. As illustrated in FIG. 4, a template nucleotide sequence 404 can be immobilized on a substrate 402 and a caged primer 406 including a cleavable or lysable caging group 408 can be hybridized to such an immobilized template. Alternatively, the template can by hybridized to a primer that does not include a caging group. A nucleotide including a caging group and complementary to the next base of the template adjacent the primer can be incorporated in situ, such as by use of polymerase, preventing further extension of the primer.

In another example illustrated in FIG. 5, a caged primer 506 including a caging group 508 can be immobilized to a substrate 502. The template nucleic acid sequence 504 can be hybridized to the caged primer 506, effectively immobilizing the nucleic acid sequence 504. In alternative examples, a template nucleotide sequence can be immobilized by rolling chain amplification techniques, polymerase chain reaction techniques, or other methods.

As illustrated at 104 of FIG. 1, a first reaction site 112 associated with a first field of view can be activated, such as through exposure to electromagnetic radiation having a wavelength effective to cleave or lyse the caging group. The field of view can be observed as synthesis proceeds within the field of view, as illustrated at 106. In particular, synthesis reactions proceed within the exposed region and not in other regions. The first reaction site 112 can be observed for a desired period of time. For example, the first reaction site can be observed until synthesis reactions slow or cease.

A second reaction site 114 associated with a different field of view can be activated, for example, exposed to electromagnetic radiation, as illustrated at 108. The field of view associated with the second reaction site 114 can be observed for synthesis, as illustrated at 110.

Alternatively or in addition, a second caged primer including a different caging group can be used. In an example, the caging group of the second caged primer can be different from the caging group of the caged primer and can respond to a different wavelength. In such an example, the first reaction site 112 can be exposed to electromagnetic radiation of a different peak wavelength, resulting in cleavage or lysing of the second caging group, activating synthesis of different templates within the first field of view. Optionally, conditions can be changed between activations of synthesis reactions. For example, the type of nutrient solution, dye labeled nucleotides, pH, temperature, or enzyme, among others, can be changed between observations of different fields of view.

In a particular example, the synthesis reaction utilizes fluorescent dye labeled nucleotides. Optionally, the polymerase incorporating the fluorescent dye labeled nucleotide to the template can include an energy donor group, such as a different dye or a quantum dot. In such an example, caged primers within the field of view can be activated using electromagnetic radiation, such ultraviolet radiation, to cleave or lyse caging groups. Nutrients, enzymes, and dye labeled nucleotides can be supplied. In addition, excitation radiation having a wavelength to energize the energy donor group can be directed toward the substrate. When a dye labeled nucleotide is incorporated, the energy donor group can donate charge to facilitate emission from the dye labeled nucleotide. Such emissions can be detected using an optical device observing the field of view. Subsequently, other fields of view can be activated and observed or the same field of view can be reactivated and observed. When a second caged primer is used, the same field of view can be observed following cleavage or lysing of the second caging group from the second caged primer.

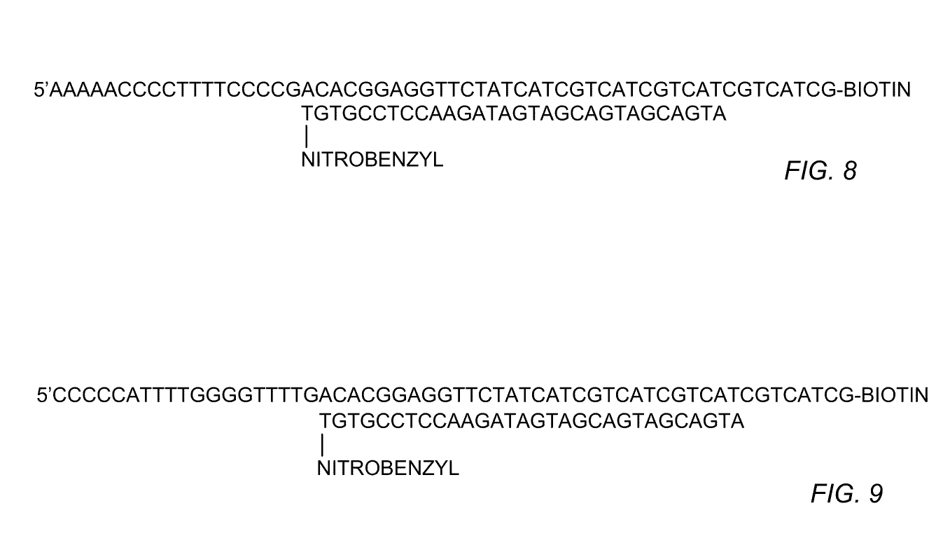

Such methods can be implemented using a sequencing device. FIG. 6 includes an illustration of an exemplary device 600. The device 600 includes a substrate 602 sized to provide multiple fields of view 604 and 606. In an example, the substrate 602 is transparent. Observation can occur from an opposite side of the substrate 602 as the synthesis reactions.

Photocleaving or photolysing can be performed using an electromagnetic radiation source 608, such as an ultraviolet radiation source. In an example, the electromagnetic radiation source 608 can be a directed energy source that can selectively address fields of view on the substrate 602. The electromagnetic radiation source 608 can be an ultraviolet radiation source having a peak wavelength in a range of 100 nm to 1000 nm, such as a range of 100 nm to 500 nm, a range of 100 nm to 400 nm, or a range of 200 nm to 360 nm. The electromagnetic radiation source 608 can selectively activate synthesis reactions within a field of view and subsequently active synthesis reactions in a different field of view.

Optionally, a second electromagnetic radiation source 610 can be included in the device 600. The second electromagnetic radiation source 610 can have a peak wavelength in the ultraviolet spectrum, such as a peak wavelength in a range of a range of 100 nm to 1000 nm, such as a range of 100 nm to 500 nm, a range of 100 nm to 400 nm, or a range of 200 nm to 360 nm. In particular, the peak wavelength of the second electromagnetic radiation source 610 can be different than the peak wavelength of the first electromagnetic radiation source 608. For example, the difference between the peak wavelengths can be at least 25 nm, such as at least 40 nm, or even at least 50 nm. The second electromagnetic radiation source 610 can be used to activate a second caged primer by cleaving or lysing the second caging group from the second caged primer.

Once the synthesis reactions are activated within a field of view, a detector 616 can be used to observe the synthesis reactions. In the context of an optically detectable synthesis reaction, for example, using dye labeled nucleotides, the detector 616 includes an optical detector, such as a photodiode, a charge coupled device, or a CMOS device. The system 600 can also include and a focusing mechanism 614. For example, when the field of view 606 is being observed, light 620 associated with fluorescent emissions is directed through the focusing mechanism 614 toward the detector 616. Alternatively, when the field of view 606 is being observed, the focusing mechanism 614 can directed light 622 associated with fluorescent emissions toward the detector 616. The focusing mechanism 614 or the detector 616 can use physical movement (e.g., changing angle or position) or can use electrooptical devices to focus on a selected field of view. Computational system 618 can receive data from the detector 616 and perform image processing, leading to sequence identification.

Optionally, the device 600 can include a further electromagnetic energy source 612 having wavelengths that energize energy donors or facilitate emission from fluorescent dye labels. In particular, electromagnetic radiation from the source 612 can be directed into the substrate 602. Techniques such as total internal reflection can be used to provide energy to those energy donors disposed near the surface of the substrate 602, limiting background noise. The energy source 612 can have a peak wavelength in a range of 380 nm to 800 nm, such as a range of 400 nm to 700 nm.

In a particular example, the substrate 602 can have one or a plurality of flow-cells. Each flow cell can include more than one field of view. For example, a single flow cell can include a plurality of fields of view that can be selectively activated by photocleaving or photolysing caged primers within the field of view. Further, the substrate 602 can define one or more fluidically-isolated wells or sites. The wells can receive DNA or compounds containing DNA. Each well can define an FOV. The substrate can receive multiple DNAs or a single DNA. Each well can also be independently activated, for example, by using UV to remove a terminator or caging group.

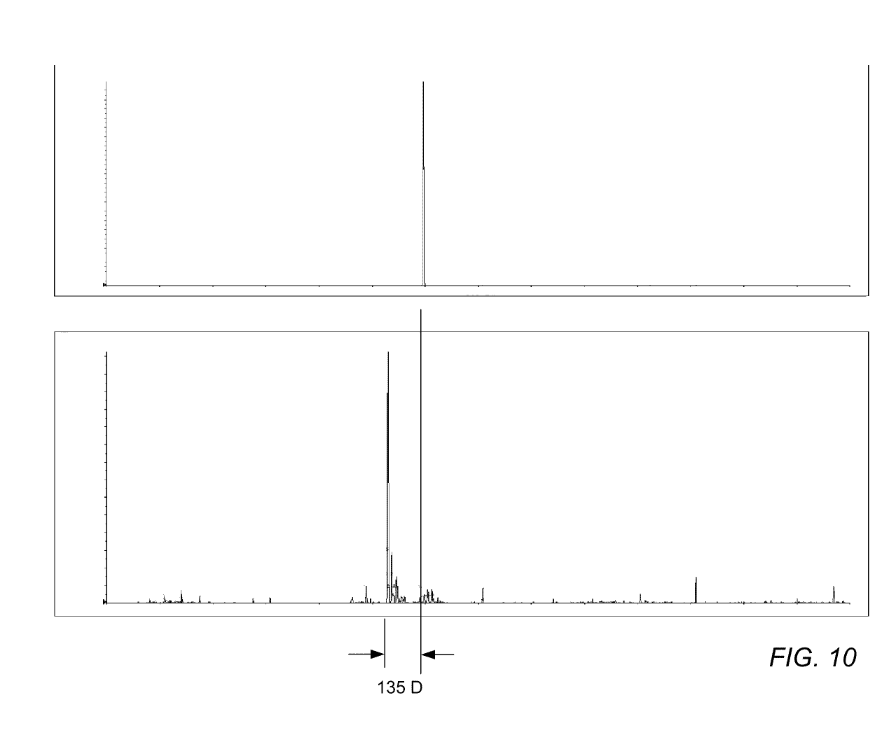

In a particular example, the substrate is a single use or limited use item. As such, the substrate can be provided as part of a kit including preparation reagents and solutions. For example, as illustrated in FIG. 7, a kit 700 includes a substrate or substrates 702. The substrates 702 can be sized to provide multiple fields of view. In a further example, the substrates 702 can define regions or wells in which templates can be immobilized and synthesis reactions can take place. Such substrates 702 can also define flow cells that can be coupled to a device to permit reagent flow over a surface of the substrate 702.

In addition, the kit 700 can include a caging group 704. The caging group 704 can be a nitrobenzyl caging group as described above. Optionally, the kit 700 can include a second caging group 706, for example, responsive to a different peak wavelength than the caging group 704. In another example, the kit 700 can include caged primers 708 or 710. The caged primers 708 or 710 can have different oligonucleotide primer, different caging groups, or a combination thereof. In another example, a nucleotide including a caging group can be included. Such a nucleotide can be used in situ to extend a primer, forming a caged or blocked primer. Further, the kit 700 can include labeled nucleotides and other reagents (not illustrated).

The above described embodiments provide desirable technical advantages. For example, a single flow cell and substrate can include multiple fields of view that are synchronously observable. Observations can be repeated in different fields of view under the same conditions or different conditions. In another example, fields of view can be sized to provide higher resolution of the observation and devices can have lower complexity. In the claims below, cleave and lyse are used interchangeably to mean both cleave and lyse.

### EXAMPLES

A caged primer is prepared that includes a nitrobenzyl group coupled to the 3-OH of an oligonucleotide primer. Exemplary template sequences are immobilized on a substrate surface using a biotin terminal group. The caged primer is hybridized to the exemplary template. Synthesis is optically observed based on fluorescence of dye labeled nucleotides.

In the examples below, the caged primer has the sequence:

5′ATGACGATGACGATGATAGAACCTCCGTGT-3′O-Nitrobenzyl

### Example 1

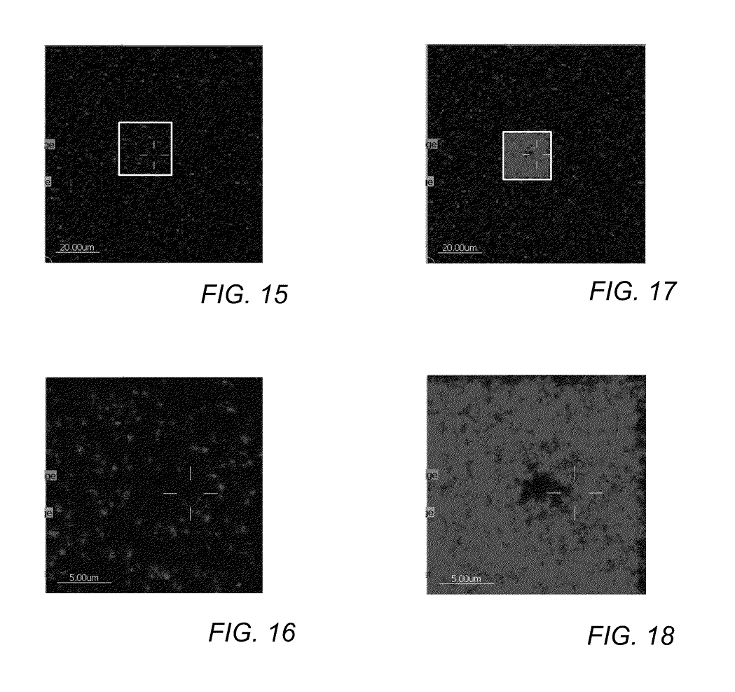

The caged primer is tested to determine responsiveness to ultraviolet radiation. Mass spectrometry is used to detect changes in molecular weight in the primer. As illustrated in FIG. 10, when the caged primer is exposed to UV radiation for 16 minutes, a shift in molecular weight of the primer of 135 units is observed. Such a shift indicates cleavage or lysing of the caging group.

### Example 2

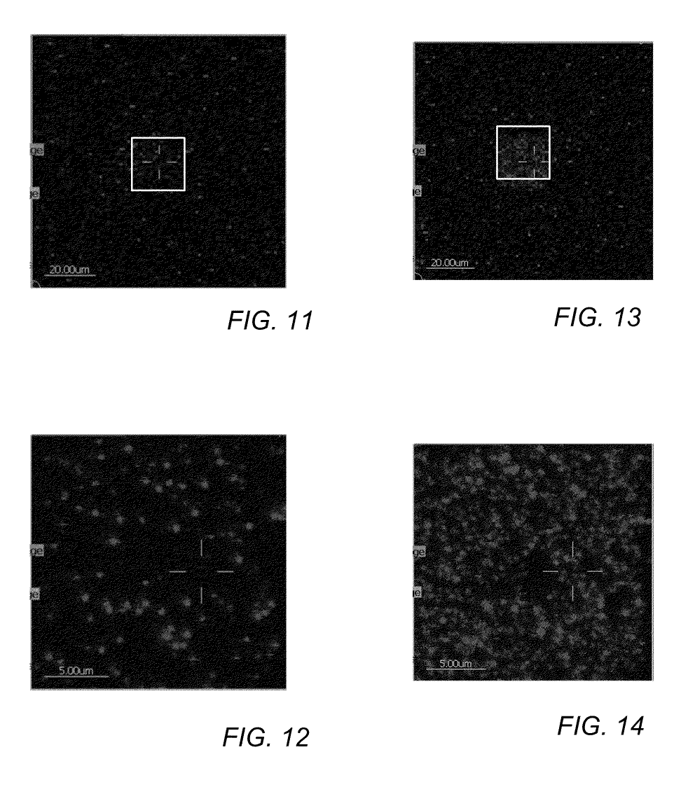

Copies of a template illustrated in FIG. 8 are attached to a substrate using a biotin terminal group. The template is applied using a solution having a template concentration of 20 pM. The caged primer is hybridized to the template as illustrated. Once synthesis is activated, the next nucleotide to be incorporated is dCTP, followed by a series of dGTP. Dye labeled dGTP (Cy5-dGTP) is used to indicate that the synthesis reaction is occurring.

The test device utilizes a confocal system to direct electromagnetic radiation at the sample. A UV 355 nm laser with objective output power of 1.75 microwatts and a 633 nm laser with objective output power of 3 microwatts are aligned. The detector includes an avalanche photodiode.

A field of view of 100 micrometers×100 micrometers is selected and scanned using the 633 nm laser. The 633 nm laser provides energy to support fluorescent emissions from the fluorescent dyes. The field of view is observed to determine that synthesis reactions are not occurring.

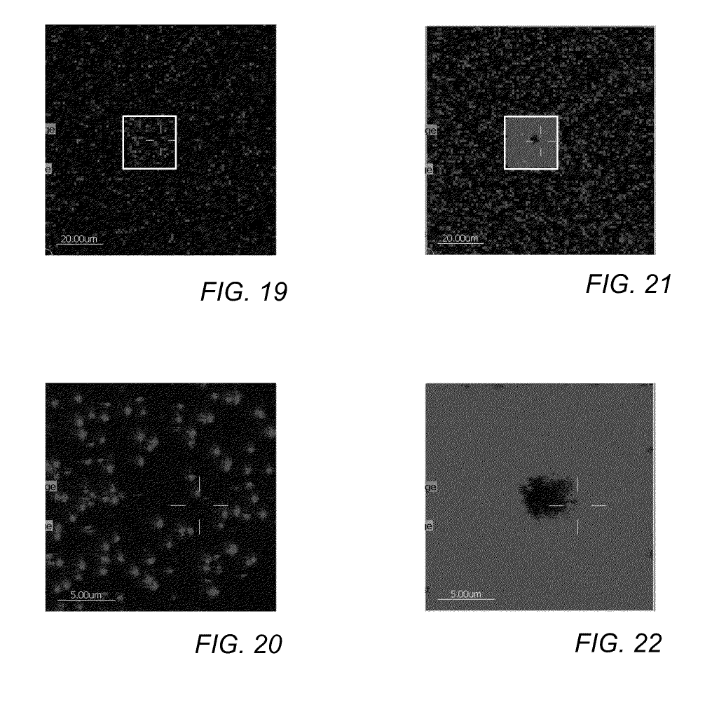

A fine scan in a 20 micrometer×20 micrometer area is performed using the UV laser source (355 nm). The sample is incubated in a solution including a Klenow enzyme, dye labeled dGTP, and Mg²⁺, without the trigger base dCTP. As illustrated in FIG. 11 and FIG. 12, few synthesis reactions are observed. FIG. 11 illustrates the 100 micrometer×100 micrometer view, and FIG. 12 illustrates the 20 micrometer×20 micrometer area scanned by the UV laser and indicated as a white box in FIG. 11. The sample is washed using 1 mL of a TBST solution.

The area is again scanned with the 633 nm laser and incubated in a solution including a Klenow enzyme, dye labeled dGTP, Mg²⁺, and the trigger base dCTP. The sample can be washed with 1 mL of a TBST solution, followed by 100 microliters of a TBST and EDTA solution. The area can be further scanned with the 633 nm laser.

As illustrated in FIG. 13 and FIG. 14, a significant number of synthesis reactions are observed within the area exposed to radiation from the UV laser. FIG. 13 illustrates the 100 micrometer×100 micrometer area, and FIG. 14 illustrates the 20 micrometer×20 micrometer area exposed to the UV laser and indicated by the white square in FIG. 13.

The example is repeated using a solution that includes the template at a concentration of 200 pM to populate the substrate. As illustrated in FIG. 15 (100 micrometer×100 micrometer) and FIG. 16 (20 micrometer×20 micrometer), the synthesis reactions are limited absent the triggering base. When the trigger base is included in solution, a significant number of synthesis reactions occur within the area exposed the UV radiation, while few reactions occur outside the exposed area, as illustrated in FIG. 17 and FIG. 18.

### Example 3

Samples using the template illustrated in FIG. 9 are prepared from a solution including the template in a concentration of 200 pM. Similar equipment and procedures are used as described above in Example 2. A trigger base dCTP is used in conjunction with dye labeled dATP (AF5-dATP).

As illustrated in FIG. 19 and FIG. 20, absent the trigger base, few synthesis reactions occur. FIG. 19 illustrates an area of 100 micrometers×100 micrometers and FIG. 20 illustrates an area of 20 micrometers×20 micrometers exposed to the UV laser and indicated by the white square of FIG. 19. When the trigger base dCTP is included in the incubating solution, synthesis reactions proceed, particularly within the area exposed to the UV laser. FIGS. 21 and 22 illustrate the fluorescent response following addition of the trigger base.

In a first aspect, a method of sequencing a plurality of template nucleotide sequences includes immobilizing the plurality of template nucleotide sequences on a substrate. A first subset of the plurality of template nucleotide sequences is immobilized in a first field of view and a second subset of the plurality of template nucleotide sequences is immobilized in a second field of view. The first and second subsets are hybridized to a caged primer. The caged primer including a caging group. The method further includes lysing the caging group from the caged primer in the first field of view and observing the first field of view to detect sequencing of the first subset of the plurality of template nucleotide sequences.

In an example of the first aspect, lysing the caging group includes photolysing the caging group from the caged primer. For example, photolysing can includes directing electromagnetic radiation having a wavelength in a range of 100 nm to 1000 nm.

In another example of the first aspect, the method further includes lysing the caging group from the caged primers in the second field of view and observing the second field of view.

In an additional example of the first aspect, a third subset of the plurality of template nucleotide sequences is hybridized to a second caged primer having a second caging group different from the caging group. The method further includes lysing the second caged group from the second caged primer. In an example, lysing the second caged group includes photolysing the second caged group. In a particular example, photolysing the second caged group includes photolysing using a peak wavelength different from a peak wavelength used during lysing the caging group.

In a further example of the first aspect, observing the first field of view includes optically detecting incorporation of nucleotides.

In another example of the first aspect, the caged primer includes nitrobenzyl functionality. In an example, the caging group is selected from the group consisting of nitrobenzyl, α-carboxy-2-nitrobenzyl (CNB), 1-(2-nitrophenyl)ethyl (NPE), 4,5-dimethoxy-2-nitrobenzyl (DMNB), 1-(4,5-dimethoxy-2-nitrophenyl)ethyl (DMNPE), 5-carboxymethoxy-2-nitrobenzyl (CMNB) caging groups, and any combination thereof. For example, the caging group can be nitrobenzyl caging group. In another example, the caging group is α-carboxy-2-nitrobenzyl (CNB) caging group. In a further example, the caging group is 1-(2-nitrophenyl)ethyl (NPE) caging group. In an additional example, the caging group is a dimethoxy caging group. For example, the dimethoxy caging group is 4,5-dimethoxy-2-nitrobenzyl (DMNB) caging group. In another example, the dimethoxy caging group is 1-(4,5-dimethoxy-2-nitrophenyl)ethyl (DMNPE) caging group.

In a second aspect, a method of sequencing a template nucleotide sequences includes applying a caged primer to the template nucleotide sequences. The caged primer includes an oligonucleotide primer and a caging group coupled to the oligonucleotide primer. The method further includes lysing the caged group from the caged primer.

In an example of the second aspect, the caging group is coupled to a terminal end of the oligonucleotide primer. For example, the terminal end is the 3′ terminal end.

In an additional example of the second aspect, the method further includes applying polymerase and a nucleotide to the template nucleotide sequences and observing to detect incorporation of the nucleotide. For example, when the nucleotide is coupled to a fluorescent dye, the fluorescent dye fluoresces in response to incorporation. Observing includes optically detecting.

In another example of the second aspect, lysing the caged group includes photolysing the caged group. For example, photolysing includes directing electromagnetic radiation toward the caged primer, the electromagnetic radiation having a wavelength in a range of 100 nm to 1000 nm.

In a further example, the caged primer includes nitrobenzyl functionality. In an example, the caging group can be selected from the group consisting of nitrobenzyl, α-carboxy-2-nitrobenzyl (CNB), 1-(2-nitrophenyl)ethyl (NPE), 4,5-dimethoxy-2-nitrobenzyl (DMNB), 1-(4,5-dimethoxy-2-nitrophenyl)ethyl (DMNPE), 5-carboxymethoxy-2-nitrobenzyl (CMNB) caging groups, and any combination thereof. For example, the caging group is a nitrobenzyl caging group. In another example, the caging group is an α-carboxy-2-nitrobenzyl (CNB) caging group. In an additional example, the caging group is an 1-(2-nitrophenyl)ethyl (NPE) caging group. In a further example, the caging group is a dimethoxy caging group. For example, the dimethoxy caging group is a 4,5-dimethoxy-2-nitrobenzyl (DMNB) caging group. In another example, the dimethoxy caging group is a 1-(4,5-dimethoxy-2-nitrophenyl)ethyl (DMNPE) caging group.

In an additional example of the second aspect, the method further includes immobilizing the template nucleotide sequences on a substrate. For example, each of the template nucleotide sequences can include a terminal group, and immobilizing can include coupling the terminal group to the substrate. In particular, the terminal group can include biotin. In another example, the caged primer is immobilized to the substrate, and immobilizing a template nucleotide sequence and applying the caged primer include coupling the template nucleotide sequence to the immobilized caged primer. For example, the caged primer can further includes a terminal group disposed on an opposite end of the caged primer from the caging group, wherein the terminal group is coupled to the substrate.

In another example of the second aspect, the method further includes applying a second caged primer to a second template nucleotide sequence. The second caged primer includes a second caging group different from the caging group. In an additional example, the caging group is responsive to a first wavelength and the second caging group is responsive to a second wavelength different from the first wavelength.

In a third aspect, a kit to sequence template nucleotide sequences includes a substrate to immobilize template nucleotide sequences. The substrate is sized to include a plurality of fields of view. The kit also includes a caged primer including an oligonucleotide primer and a caging group coupled to a terminal end of the oligonucleotide primer.

In an example of the third aspect, the terminal end is the 3′ terminal end. In another example of the third aspect, the substrate is a transparent substrate.

In an additional example of the third aspect, the kit further includes a second caged primer including a second caging group. For example, the caging group is responsive to a first wavelength and the second caging group is responsive to a second wavelength different from the first wavelength.

In a further example, the kit includes a caging nucleotide.

In a fourth aspect, a device includes a substrate to immobilize a plurality of target nucleotides. A target nucleotide of the plurality of target nucleotides is hybridized to a caged primer. The caged primer includes a caging group. The substrate is sized to provide a plurality of fields of view. The device further includes a radiation source to selectively address fields of view of the plurality of fields of view. The radiation source has a peak wavelength to lyse the caging group from the caged primer in a select field of view of the plurality of fields of view. The device also includes a detector to detect incorporation of a nucleotide to the target nucleotide in the select field of view.

In an example of the fourth aspect, the detector includes an optical detector and a focusing mechanism to collect emission radiation from the select field of view.

In another example of the fourth aspect, the device further includes a second radiation source. For example, the second radiation source is to selectively address fields of view of the plurality of fields of view. In another example, the second radiation source is to selectively address the select field of view of the plurality of fields of view, the second radiation source having a second peak wavelength different from the peak wavelength of the radiation source. In an additional example, wherein the nucleotide includes a fluorescent dye, the second radiation source is to provide energy to facilitate emission of the fluorescent dye in response to incorporation of the nucleotide.

In a fifth aspect, a method of sequencing a template nucleotide sequence includes applying a primer to the template nucleotide sequence. The primer includes an oligonucleotide hybridized to a portion of the template nucleotide sequence. The method further includes incorporating a caging nucleotide including a caging group to a terminal end of the oligonucleotide hybridized to the portion of the template nucleotide.

In an example of the fifth aspect, the method further includes lysing the caging group from the caging nucleotide following incorporating and observing the template nucleotide sequence. For example, lysing the caged group includes photolysing the caged group. In an example, photolysing includes directing electromagnetic radiation toward the caging group. The electromagnetic radiation has a wavelength in a range of 100 nm to 1000 nm.

In another example of the fifth aspect, the caging group includes nitrobenzyl functionality. For example, the caging group can be selected from the group consisting of nitrobenzyl, α-carboxy-2-nitrobenzyl (CNB), 1-(2-nitrophenyl)ethyl (NPE), 4,5-dimethoxy-2-nitrobenzyl (DMNB), 1-(4,5-dimethoxy-2-nitrophenyl)ethyl (DMNPE), 5-carboxymethoxy-2-nitrobenzyl (CMNB) caging groups, and any combination thereof. In an example, the caging group is a nitrobenzyl caging group. In another example, the caging group is an α-carboxy-2-nitrobenzyl (CNB) caging group. In an additional example, the caging group is an 1-(2-nitrophenyl)ethyl (NPE) caging group. In a further example, the caging group is a dimethoxy caging group. For example, the dimethoxy caging group can be a 4,5-dimethoxy-2-nitrobenzyl (DMNB) caging group. In another example, the dimethoxy caging group is a 1-(4,5-dimethoxy-2-nitrophenyl)ethyl (DMNPE) caging group.

In a further example of the fifth aspect, the method further includes immobilizing the template nucleotide sequences on a substrate. In an example, each of the template nucleotide sequence includes a terminal group, and immobilizing includes coupling the terminal group to the substrate. In a particular example, the terminal group includes biotin. In an additional example, the primer is immobilized to the substrate, and immobilizing a template nucleotide sequence and applying the primer include coupling the template nucleotide sequence to the immobilized primer.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

1. A method of sequencing a plurality of template nucleotide sequences, the method comprising:
immobilizing the plurality of template nucleotide sequences on a substrate, a first subset of the plurality of template nucleotide sequences immobilized in a first field of view and a second subset of the plurality of template nucleotide sequences immobilized in a second field of view, the first and second subsets hybridized to a caged primer, the caged primer including a caging group;
lysing the caging group from the caged primer in the first field of view; and
observing the first field of view to detect sequencing of the first subset of the plurality of template nucleotide sequences.

2. The method of claim 1, wherein lysing the caging group includes photolysing the caging group from the caged primer.

3. The method of claim 2, wherein photolysing includes directing electromagnetic radiation having a wavelength in a range of 100 nm to 1000 nm.

4. The method of claim 1, further comprising lysing the caging group from the caged primers in the second field of view and observing the second field of view.

5. The method of claim 1, wherein a third subset of the plurality of template nucleotide sequences is hybridized to a second caged primer having a second caging group different from the caging group, the method further comprising lysing the second caged group from the second caged primer.

6. The method of claim 5, wherein lysing the second caged group includes photolysing the second caged group.

7. The method of claim 6, wherein photolysing the second caged group includes photolysing using a wavelength different from a wavelength used during lysing the caging group.

8. The method of claim 1, wherein observing the first field of view includes optically detecting incorporation of nucleotides.

9. (canceled)

10. The method of claim 1, wherein the caging group is selected from the group consisting of nitrobenzyl, α-carboxy-2-nitrobenzyl (CNB), 1-(2-nitrophenyl)ethyl (NPE), 4,5-dimethoxy-2-nitrobenzyl (DMNB), 1-(4,5-dimethoxy-2-nitrophenyl)ethyl (DMNPE), 5-carboxymethoxy-2-nitrobenzyl (CMNB) caging groups, and any combination thereof.

11. The method of claim 10, wherein the caging group is a nitrobenzyl caging group.

12. (canceled)

13. (canceled)

14. The method of claim 10, wherein the caging group is a dimethoxy caging group.

15-16. (canceled)

17. A method of sequencing a template nucleotide sequences, the method comprising:
applying a caged primer to the template nucleotide sequences, the caged primer including an oligonucleotide primer and a caging group coupled to the oligonucleotide primer; and
lysing the caged group from the caged primer.

18. The method of claim 17, wherein the caging group is coupled to a terminal end of the oligonucleotide primer.

19. The method of claim 18, wherein the terminal end is the 3′ terminal end.

20. The method of claim 17, further comprising applying polymerase and a nucleotide to the template nucleotide sequences and observing to detect incorporation of the nucleotide.

21. The method of claim 20, wherein the nucleotide is coupled to a fluorescent dye, the fluorescent dye fluorescing in response to incorporation, and wherein observing includes optically detecting.

22. The method of claim 17, wherein lysing the caged group includes photolysing the caged group.

23. (canceled)

24. The method of claim 17, wherein the caged primer includes nitrobenzyl functionality.

25-31. (canceled)

32. The method of claim 17, further comprising immobilizing the template nucleotide sequences on a substrate.

33-38. (canceled)

39. A kit to sequence template nucleotide sequences, the kit comprising:
a substrate to immobilize template nucleotide sequences, the substrate sized to include a plurality of fields of view; and
a caged primer including an oligonucleotide primer and a caging group coupled to a terminal end of the oligonucleotide primer.

40-65. (canceled)


SEQUENCE LISTING (3 sequences)

SEQ ID NO: 1 - DNA, 30 bp, Artificial Sequence
  Primer complementary to template of know sequence to test sequencing methods
atgacgatgacgatgatagaacctccgtgt

SEQ ID NO: 2 - DNA, 56 bp, Artificial Sequence
  Known sequence to test performance of sequencing methods.
aaaaaccccttttccccgacacggaggttctatcatcgtcatcgtcatcgtcatcg

SEQ ID NO: 3 - DNA, 57 bp, Artificial Sequence
  Known sequence to test performance of sequencing methods
cccccattttggggttttgacacggaggttctatcatcgtcatcgtcatcgtcatcg